(19) United States
(12) Patent Application Publication
(10) Pub. No.: US 2014/0194745 A1
(43) Pub. Date: Jul. 10, 2014

(54) ULTRASOUND IMAGE DIAGNOSTIC DEVICE

(71) Applicant: KONICA MINOLTA, INC., Tokyo (JP)

(72) Inventor: Tsuyoshi OKUBO, Tokyo (JP)

(73) Assignee: KONICA MINOLTA, INC., Tokyo (JP)

(21) Appl. No.: 14/208,230

(22) Filed: Mar. 13, 2014

Related U.S. Application Data
(62) Division of application No. 13/487,599, filed on Jun. 4, 2012.

(30) Foreign Application Priority Data
Jun. 14, 2011 (JP) 2011-131755
Sep. 7, 2011 (JP) 2011-194906

(51) Int. Cl. A61B 8/08; A61B 8/00

(52) U.S. Cl. CPC A61B 8/5269; A61B 8/4483
USPC 600/459

(57) ABSTRACT

An ultrasound image diagnostic device including: a transducer to output transmission ultrasound toward a subject by a drive signal and also to output a reception signal by receiving reflection ultrasound from the subject; a noise output section in which when the transducer receives the reflection ultrasound, a voltage containing noise is applied to the transducer to amplify the reception signal by a stochastic resonance phenomenon; a harmonic extracting section to extract a harmonic component from the reception signal; and an image processing section to generate ultrasound diagnostic image data of an interior of the subject based on the harmonic component extracted by the harmonic extracting section, wherein the noise output section applies a voltage containing noise such that the harmonic component is amplified by the stochastic resonance phenomenon to the transducer.

This is a Divisional of U.S. application Ser. No. 13/487,599, filed Jun. 4, 2012, which is based on Japanese Patent Application Nos. 2011-131755, filed Jun. 14, 2011, and 2011-194906, filed Sep. 7, 2011, the entire contents of all of which are incorporated herein by reference.

### TECHNICAL FIELD

The present invention relates to an ultrasound image diagnostic device.

### BACKGROUND

Over recent years, medical diagnostic methods have been known in which ultrasound is transmitted to a subject; reflection ultrasound generated via reflection/scattering of transmission ultrasound at boundaries where there is a difference in acoustic impedance is received; and then on the basis of this reception signal, imaging is carried out to observe the form of a living body non-invasively. In such medical diagnostic methods, for example, ultrasound image diagnostic devices are used. In these ultrasound image diagnostic devices, there are used piezoelectric materials capable of converting an electrically produced transmission waveform into sound pressure (transmission ultrasound) and also of converting reflection ultrasound into an electrical signal.

According to the above principle, with an increase in the frequency of transmission ultrasound, an image having enhanced spatial resolution can be obtained. On the other hand, there is large attenuation in the interior of a subject and then the intensity of reflection ultrasound decreases, resulting in a decrease in S/N (Signal-Noise ratio). In other words, the ratio of noise contained in a reception signal increases and then there occurs a decrease in dynamic range. Herein, the dynamic range is defined by subtracting the level of noise from the maximum signal level. Further, the intensity of reflection ultrasound decreases with the depth inside a subject. Thereby, a technique to enhance the S/N of a reception signal, with satisfaction of any of the factors contrary to each other such as image quality, frequency, and depth has been a problem. Therefor, various improvements have been made with respect to transducers to transmit and receive ultrasound, electrical amplification techniques for reception signals, signal processing, and image processing.

For example, as employed piezoelectric materials, PZT (zirconate-titanate solid solution) and P(VDF-3FE) (polyvinylidene fluoride-polytrifluoroethylene copolymer) are known. The former exhibits enhanced sound pressure-electricity conversion efficiency, being used in many cases to obtain a reception signal of enhanced sensitivity and S/N. In contrast, the latter is inferior to the former in sound pressure-electricity conversion efficiency but has large sensitive frequency bandwidth, being therefore applied in some cases to ultrasound image diagnostic devices handling reception signals of high frequency and broadband.

However, new piezoelectric materials to enhance sound pressure-electricity conversion have been so far developed, but there have been found out no materials having piezoelectricity/frequency characteristics exceeding the performance of PZT and P(VDF-3FE).

Further, as a technique widely used to electrically amplify a reception signal, there is known an amplifier such as a TGC (Time Gain Compensation) amplifier to change the gain of a signal based on the depth of a diagnostic object. This is an extremely-low noise amplifier in which depth is converted into time from sound velocity and the depth of an echo source is measured by the elapsed time just after transmitting ultrasound to change the gain of a reception signal based on the thus-measured elapsed time in order thereby to enhance S/N.

However, in the case of use of such an amplifier, not only a signal but also noise are amplified, resulting in a limit in S/N enhancement. Further, the amplifier generates thermal electrons by being driven and thereby noise is generated. The influence of such noise is not negligible. Incidentally, a technique to cool an amplifier in order to reduce the generation of thermal electrons has been also known. However, since thermal electrons cannot be completely prevented from being generated, there is a limit in S/N enhancement after all. Further, an apparatus to cool the amplifier causes the size increase of the device, resulting also in cost increase.

Further, as a method to enhance S/N by amplifying a reception signal via signal processing, a coding technique has been known. This is a technique in which a transmission signal to output transmission ultrasound is subjected to phase or frequency modulation; a reception signal and the transmission signal are autocorrelated; and then only an extracted signal is imaged. According to this technique, S/N increases depending on the number of phase-modulated signals for the phase modulation and on the frequency shift amount for the frequency modulation.

Further, in a recent ultrasound image diagnostic device, an ultrasound probe, in which a plurality of piezoelectric elements are arranged in an array manner, is used. A reception signal from each element is subjected to summing after aligning the phase of the reception signal and then noise contained in the reception signal is reduced, whereby S/N enhancement can be realized by the averaging principle. Further, according to this technique, with an increase in the number of elements (n), noise is suppressed by 1/(n^{1/2}), resulting further in an expectation for S/N enhancement.

Still further, when the same averaging principle is applied to interframe integration for image processing (frame averaging), S/N enhancement can also be realized.

However, according to the coding technique, in a living body, ultrasound is reflected/scattered in a complex manner due to the characteristics of body tissues and further reflection ultrasound having high frequency is attenuated to a large extent, whereby in the course of demodulation of a reception signal, an unintended signal is amplified and then a strong sidelobe is expressed, resulting in the possibility of a decrease in S/N. On the other hand, when for large gain and sidelobe suppression, coding length and coding dimension are increased, pulse width and the demodulation time of a reception signal are increased, resulting in the problem of the decrease of axial resolution and frame rate.

Further, according to beam forming and interframe integration associated with an increase in the number of elements, the needed amount of operations increases and also a decrease in frame rate is unavoidable.

Of the above-described techniques taking countermeasures against the problem to enhance the S/N of a reception signal, there is disclosed a technique in which in a conventional ultrasound image diagnostic device, the interior of a subject is irradiated with light and then an optoacoustic wave having been generated by this light irradiation is received by a piezoelectric element to generate an ultrasound diagnostic image based thereon. In this ultrasound image diagnostic device, since the intensity of a reception signal acquired from such a photoacoustic wave is extremely low, a stochastic resonance method is applied for S/N enhancement, in which noise is added to a piezoelectric element to amplify a reception signal via stochastic resonance. According to this technique, since the pulse length of an acoustic wave generated via irradiation of light having short-pulse characteristics is also short, excellent time resolution can be expressed and also enhanced spatial resolution can be realized (for example, Unexamined Japanese Patent Application Publication No. 2009-165634).

However, in the ultrasound image diagnostic device described in Unexamined Japanese Patent Application Publication No. 2009-165634 (Patent document 1), the penetration depth of light is extremely small and thereby an acoustic wave up to a depth of at most about 6 mm can be merely obtained.

Further, in the technique described in above Patent Document 1, with respect to an electrical signal having small intensity, S/N is expected to be improved to some extent, but since the amplification amount of the signal is at a certain degree, the effect is limited.

An object of the present invention is to provide an ultrasound image diagnostic device in which an excellent ultrasound diagnostic image can be obtained by a reception signal having enhanced spatial resolution in large depth and enhanced S/N.

Another object of the present invention is to provide a piezoelectric sensor having improved S/N in an electrical signal in which the intensity generated based on the stress of a piezoelectric material is low, an ultrasound probe, and an ultrasound image diagnostic device.

### SUMMARY

To solve the above problems, in the invention described in item 1, an ultrasound image diagnostic device comprises:

a transducer to output transmission ultrasound toward a subject by a drive signal and also to output a reception signal by receiving reflection ultrasound from the subject;

a noise output section in which when the transducer receives the reflection ultrasound, a voltage containing noise is applied to the transducer to amplify the reception signal by a stochastic resonance phenomenon;

a harmonic extracting section to extract a harmonic component from the reception signal; and

an image processing section to generate ultrasound diagnostic image data of an interior of the subject based on the harmonic component extracted by the harmonic extracting section,

wherein the noise output section applies a voltage containing noise such that the harmonic component is amplified by the stochastic resonance phenomenon to the transducer.

In the invention described in item 2, in the ultrasound image diagnostic device described in item 1,

the noise output section applies a voltage containing noise in which a third harmonic component is amplified by the stochastic resonance phenomenon to the transducer.

In the invention described in item 3, in the ultrasound image diagnostic device described in item 1,

the noise output section applies the voltage containing the noise to the transducer so that on the basis of the depth of the reflection ultrasound received by the transducer, the gain of the amplification of the harmonic component by the stochastic resonance phenomenon is changed per harmonic order.

In the invention described in item 4, in the ultrasound image diagnostic device described in item 3,

the noise output section changes the pattern of noise contained in an applied voltage to change, per harmonic order, the gain of the amplification of the harmonic component by the stochastic resonance phenomenon.

In the invention described in item 5, in the ultrasound image diagnostic device described in item 1,

the noise output section applies the voltage containing noise such that the harmonic component is amplified at the timing when the transducer receives reflection ultrasound from a predetermined depth in the subject to the transducer.

In the invention described in item 6, the ultrasound image diagnostic device described in item 1 is provided with

a bias voltage feeding section in which a bias voltage is superimposed to a voltage output by the noise output section so that the voltage is matched to the baseline of the reception signal.

In the invention described in item 7, in the ultrasound image diagnostic device described in item 6,

the bias voltage feeding section changes the magnitude of the bias voltage to be superimposed based on the depth of the reflection ultrasound received by the transducer.

In the invention described in item 8, an ultrasound image diagnostic device is provided with a transducer having:

a piezoelectric member in which a primary side section and a secondary side section are formed in a longitudinal direction, the primary side section is polarized in a thickness direction, and the secondary side section is polarized in the longitudinal direction;

a primary side electrode formed on a face intersecting with the thickness direction of the primary side section of the piezoelectric member; and

a secondary side electrode formed on an end face intersecting with the longitudinal direction of the secondary side section of the piezoelectric member,

wherein when a drive signal is provided for the primary side electrode, the piezoelectric member is oscillated to output transmission ultrasound toward a subject and when stress is added to the piezoelectric member by reflection ultrasound from the subject, a reception signal in response to the added stress is output from the secondary side electrode;

a noise output section connected to the primary side electrode to amplify the reception signal by a stochastic resonance phenomenon, in which when stress is added to the piezoelectric member by the reflection ultrasound, a voltage containing noise is applied to the primary side electrode; and

an image processing section to generate ultrasound diagnostic image data of an interior of the subject based on the reception signal.

In the invention described in item 9, the ultrasound image diagnostic device described in item 8 is provided with

a harmonic extracting section to extract a harmonic component from the reception signal, wherein the image processing section generates ultrasound diagnostic image data of the interior of the subject based on a harmonic component extracted by the harmonic extracting section and the noise output section applies a voltage containing noise such that the harmonic component is amplified by the stochastic resonance phenomenon to the primary side electrode.

In the invention described in item 10, in the ultrasound image diagnostic device described in item 9, wherein the noise output section applies the voltage containing noise such that the third harmonic component is amplified by the stochastic resonance phenomenon to the primary side electrode.

In the invention described in item 11, in the ultrasound image diagnostic device described in item 9, wherein the noise output section applies a voltage containing noise to the primary side electrode so that gain of an amplification of the harmonic component by the stochastic resonance phenomenon is changed per harmonic order based on the depth of reflection ultrasound adding stress to the piezoelectric member.

In the invention described in item 12, in the ultrasound image diagnostic device described in item 11, wherein the noise output section changes the pattern of noise contained in a voltage applied to the primary side electrode to change, per harmonic order, the gain of the amplification of the harmonic component by the stochastic resonance phenomenon.

In the invention described in item 13, in the ultrasound image diagnostic device described in item 9, wherein the noise output section applies a voltage containing noise such that the harmonic component is amplified at the timing when stress is added to the piezoelectric member by reflection ultrasound from a predetermined depth in the subject to the primary side electrode.

### BRIEF DESCRIPTION OF THE DRAWINGS

FIG. 1 is a view showing the external configuration of an ultrasound image diagnostic device;

FIG. 2 is a block diagram showing the schematic configuration of the ultrasound image diagnostic device;

FIG. 3 is a view illustrating the signal levels of a signal component and a noise component;

FIG. 4 is a view illustrating the relationship between depth and bias voltage;

FIG. 5 is a schematic view representing the frequency spectrum of reflection ultrasound;

FIG. 6 is a view illustrating the relationship between signal and noise components and depth;

FIG. 7 is a schematic view illustrating the S/N of a reception signal in the case of no application of a stochastic resonance phenomenon; and

FIG. 8 is a schematic view illustrating the S/N of a reception signal in the case of application of a stochastic resonance phenomenon.

FIG. 9 is a schematic cross-sectional view showing the configuration of an ultrasound probe;

FIG. 10 is a view illustrating the structure of a transducer;

FIG. 11 is a graph showing the relationship between the sound pressure of received reflection ultrasound and the S/N of a reception signal; and

FIG. 12 is a graph showing the temporal change of the signal intensity acquired by the transducer.

### PREFERRED EMBODIMENTS OF THE INVENTION

An ultrasound image diagnostic device according to a first embodiment of the present invention will now be described with reference to the drawings. However, the scope of the invention is not limited to the illustrated examples. Herein, in the following description, the same symbols are assigned to those having the same functions and configurations and the description thereof will be omitted.

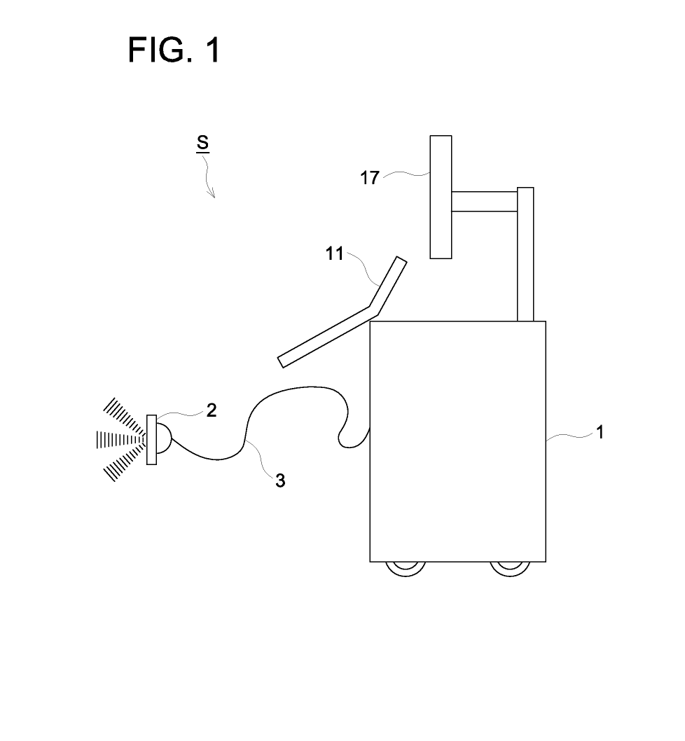

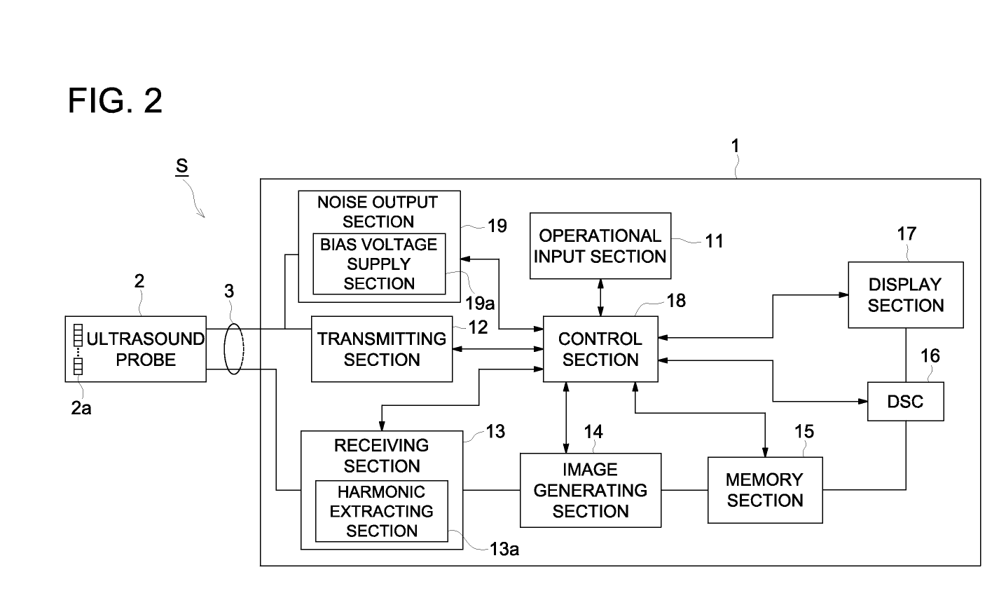

The ultrasound image diagnostic device S according to the first embodiment is provided, as shown in FIG. 1 and FIG. 2, with an ultrasound image diagnostic device main body 1 and an ultrasound probe 2. The ultrasound probe 2 transmits ultrasound (transmission ultrasound) to an unshown subject such as a living body and also receives an ultrasound reflection wave (reflection ultrasound: echo) having been reflected by this subject. The ultrasound image diagnostic device main body 1, connected to the ultrasound probe 2 via a cable 3, transmits a drive signal of an electrical signal to the ultrasound probe 2 to allow the ultrasound probe 2 to transmit transmission ultrasound to a subject and also to image the internal state of the subject as an ultrasound image based on a reception signal of an electrical signal having been generated by the ultrasound probe 2 in response to reflection ultrasound from the interior of the subject which has been received by the ultrasound probe 2.

The ultrasound probe 2 is provided with a transducer 2a containing a piezoelectric element. A plurality of the above transducers 2a are arranged, for example, in a one-dimensional array manner in the azimuth direction. In the first embodiment, for example, an ultrasound probe 2 provided with 192 transducers 2a is used. Herein, the transducers 2a may be arranged in a two-dimensional array manner. Further, the number of the transducers 2a can be set appropriately. Still further, in the first embodiment, for the ultrasound probe 2, a linear scanning-type electronic scan probe was employed but any of an electronic scanning type and a mechanical scanning type is employable. And, any of a linear scanning type, a sector scanning type, and a convex scanning type may also be employed. Further, in the first embodiment, an employed piezoelectric element applied to the transducer 2a is PZT or P (VDF-3FE) with no limitation thereto.

The ultrasound image diagnostic device main body 1 is configured in such a manner that as shown in FIG. 2, for example, an operation input section 11, a transmitting section 12, a receiving section 13, an image generating section 14, a memory section 15, a DSC (Digital Scan Converter) 16, a display section 17, a control section 18, and a noise output section 19 are provided.

The operation input section 11 is provided with, for example, various types of switch, button, track ball, mouse, and keyboard to input commands to instruct the diagnosis initiation and data such as personal information of a subject to output an operation signal to the control section 18.

The transmitting section 12 is a circuit, in which in accordance with the control of the control section 18, a drive signal being an electrical signal is fed to the ultrasound probe 2 via the cable 3 to allow the ultrasound probe 2 to generate transmission ultrasound. Further, the transmitting section 12 is provided with, for example, a clock generating circuit, a delay circuit, and a pulse generating circuit. The clock generating circuit is a circuit to generate a clock signal to determine the transmission timing and the transmission frequency of a drive signal. The delay circuit is a circuit in which with regard to the transmission timing of a drive signal, delay time is set per individual channel corresponding to each transducer 2a and then the transmission of the drive signal is delayed by the thus-set delay time to converge transmission beams containing transmission ultrasound. The pulse generating circuit is a circuit to generate a pulse signal as the drive signal at a predetermined period.

The transmitting section 12 configured in the above manner sequentially switches a plurality of transducers 2a feeding drive signals with shifting of a predetermined number thereof per transmission/reception of ultrasound in accordance with the control of the control section 18, and then the drive signals are fed to a plurality of the transducers 2′a selected for outputting to carry out scanning.

The receiving section 13 is a circuit to receive a reception signal of an electrical signal via the cable 3 from the ultrasound probe 2 in accordance with the control of the control section 18. The receiving section 13 is provided with, for example, an amplifier, an A/D conversion circuit, and a beam forming circuit. The amplifier is a circuit to amplify a reception signal with respect to an individual channel corresponding to each transducer at a given gain preset. The A/D conversion circuit is a circuit to A/D-convert an amplified reception signal. The beam forming circuit is a circuit to provide an A/D-converted reception signal with a delay time with respect to an individual channel corresponding to each transducer 2a for phasing, followed by summing thereof (phasing and summing) to generate beam data.

Further, the receiving section 13 is provided with a harmonic extracting section 13a to eliminate the fundamental component of a reception signal and to extract harmonic components such as a second harmonic component and a third harmonic component which are frequency components of integral multiples of the fundamental component.

Herein, the receiving section 13 may be provided with a noise elimination filter to eliminate, from a reception signal, noise components having been output by a noise output section 19 to be described later.

The image generating section 14, as the image processing section, carries out envelope detection processing and logarithmic amplification for beam data from the receiving section 13 and brightness conversion via gain adjustment to generate B-mode image data. That is, the B-mode image data represents the intensity of a reception signal by brightness.

The memory section 15 contains, for example, a semiconductor memory such as a DRAM (Dynamic Random Access Memory) and memorizes, on a frame unit basis, B-mode image data having been transmitted from the image generating section 14. Namely, the memory section 15 can carry out memorizing as ultrasound diagnostic image data configured based on a frame unit basis. Then, the thus-memorized ultrasound diagnostic image data are transmitted to the DSC 16 in accordance with the control of the control section 18.

The DSC 16 converts ultrasound diagnostic image data received by the memory section 15 into an image signal based on the scanning system of television signals to be output to the display section 17.

As the display section 17, applicable is a display device such as an LCD (Liquid Crystal Display), a CRT (Cathode-Ray Tube) display, an organic EL (Electronic Luminescence) display, an inorganic EL display, or a plasma display. The display section 17 displays an ultrasound diagnostic image on the display screen in response to an image signal output from the DSC 16. Herein, instead of the display device, a printing device such as a printer may be applied.

The control section 18 is constituted of, for example, a CPU (Central Processing Unit), a ROM (Read Only Memory), and a RAM (Random Access Memory), reading out various types of processing program such as a system program memorized in the ROM to be developed on the RAM for central controlling of the operation of each section of the ultrasound image diagnostic device S in accordance with the developed program.

The ROM contains a nonvolatile memory such as a semiconductor and memorizes a system program corresponding to the ultrasound image diagnostic device S and various types of processing program executable on the system program, as well as various types of data. These programs are stored in the form of a program code which can be read by the computer, and the CPU sequentially executes operations in accordance with the program code.

The RAM forms a work area to temporarily memorize various types of program executed by the CPU and data relevant to these programs.

The noise output section 19 is provided with, for example, a timing clock and an oscillator and applies a small voltage containing noise to the transducer 2a in accordance with the control from the control section 18 at the timing of reception of reflection ultrasound by the transducer 2a. When applied with a small voltage from the noise output section 19, the transducer 2a is minutely deformed in response to noise contained in the small voltage. When the transducer 2a is applied with a small voltage and receives reflection ultrasound, the deformation of the transducer 2a is resonated at a certain frequency by a stochastic resonance phenomenon to amplify the strain with respect to the reception of reflection ultrasound. Therefore, the intensity of a converted reception signal is amplified to enhance S/N. Herein, the timing of application of a small voltage to the transducer 2a may be followed by the timing of reception of reflection ultrasound. Further, at the reception timing of reflection ultrasound from a predetermined depth, application of a small voltage to the transducer 2a may be initiated. For example, at the reception timing of reflection ultrasound from the depth where a third harmonic component is generated, application of a low voltage may be initiated.

As a noise component output from the noise output section 19, either of white noise and colored noise is applicable. For example, as the colored noise, pink noise, blue noise, and violet noise are cited. The white noise is noise in which power spectrum density is constant regardless of frequency. The pink noise is noise in which power spectrum density is inversely proportional to frequency. The blue noise is noise in which power spectrum density is proportional to frequency. The violet noise is noise in which power spectrum density is proportional to the square of frequency. Herein, the output noise component is not limited to the above ones and any appropriate power spectrum density is employable. For example, a noise component, in which the power spectrum density is one such that a stochastic resonance phenomenon readily occurs with respect to a second harmonic component and a third harmonic component, may be output.

Herein, reflection ultrasound in a subject provides a complex spectrum whose band and amplitude are further modulated with respect to transmission ultrasound. Therefore, in order to allow the response of the transducer 2a to such reflection ultrasound to resonate with high probability and to allow this phenomenon to be applied also in any depth, the noise with an optimized frequency component and intensity by time is superimposed to the transducer 2a. Especially, in a deep portion, since the intensity of a reception signal obtained from reflection ultrasound is very weak, the reception signal is desirably amplified effectively by the stochastic resonance phenomenon.

In the first embodiment, attention is focused on the fact that a frequency component amplified via the stochastic resonance phenomenon varies with the kind of noise. And then, the noise output section 19 optimizes noise contained in a small voltage applied to the transducer 2a based on the depth of received reflection ultrasound and thereby intensity decreases based on the harmonic order, whereby S/N enhancement is realized also with respect to a harmonic component having wider bandwidth in the same manner as the fundamental component. For example, the noise output section 19 changes the pattern of noise contained in a small voltage applied to the transducer 2a based on the depth of received reflection ultrasound to change the gain of a harmonic component per harmonic order based on the depth. Noise superimposed to the transducer 2a to generate a stochastic resonance phenomenon can be optimized by allowing the frequency bandwidth (BW_{N}) and the amplitude (A_{N}) of noise and the timing of superimposing noise (T_{N}) to be variable based on the central frequency (f_{R}), the frequency bandwidth (BW_{R}), the signal level (V_{R}), and the intensity (I_{R}) of a reception signal to be amplified.

Herein, noise optimization based on the depth is not limited to one realized through the change of the pattern of noise described above and may be ones realized by other methods. For example, it is possible to realize noise optimization based on the depth by changing output parameters of a small voltage.

As a preferred embodiment in the first embodiment, for example, one satisfying the following Expressions (1) and (2) is cited.

BW_{N}=(10⁻³−10⁻⁴)f_{R} (1)

A_{N}=(10⁻¹²−10²)I_{R} (2)

Therefore, when a reception signal is broadband, the frequency bandwidth (BW_{N}) of noise is adjusted, and thereby a reception signal of a desired bandwidth can be amplified and then a filter circuit such as a bandpass filter, a high-pass filter, and a low-pass filter having the same function may be unnecessary.

Further, when the timing (T_{N}) of superimposing noise is adjusted, reflection ultrasound from a predetermined depth can be selectively amplified, and thereby a configuration such as TGC to change the gain of a reception signal based on the depth may be unnecessary.

In the first embodiment, especially when reflection ultrasound is received from a deep portion, the noise output section 19 is controlled to provide the transducer 2a with noise in which a third harmonic component is amplified.

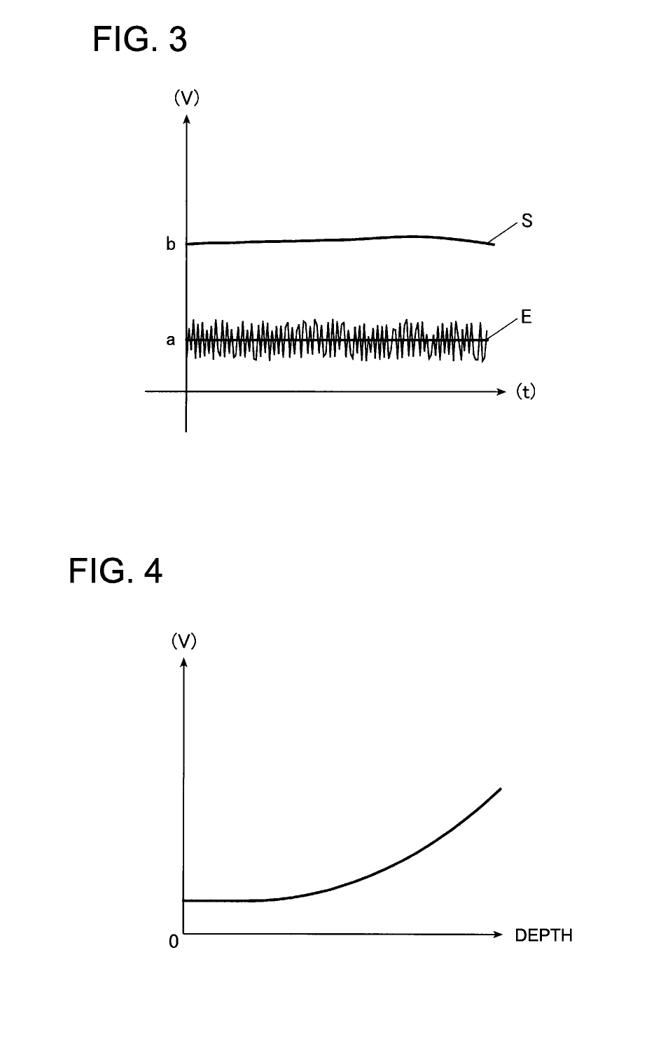

Further, the noise output section 19 is provided with a bias voltage supply section 19a and amplifies a voltage by applying a bias to a small voltage output. The bias voltage supply section 19a amplifies a small voltage so as to become a voltage corresponding to the baseline of a reception signal converted from reflection ultrasound by the transducer 2a. The reason is that to effectively generate a stochastic resonance phenomenon, a voltage containing noise needs to be matched to a voltage corresponding to the baseline of a signal causing a stochastic resonance phenomenon. Specifically, as described below, bias setting is carried out. That is, for example, as shown in FIG. 3, when the reference voltage of a small voltage E output from the noise output section 19 is aV and a voltage corresponding to the baseline of a reception signal S is bV, the voltage difference therebetween (ΔV) is represented by following Expression (3).

ΔV=bV−aV (3)

Then, the bias voltage supply section 19a superimposes a bias of a voltage corresponding to a voltage difference (ΔV) determined in such a manner to a small voltage output from the noise output section 19.

Further, a voltage corresponding to the baseline of a reception signal increases based on the depth. Therefore, a bias superimposed to a small voltage also needs to follow this. In the first embodiment, the bias voltage supply section 19a is provided with, for example, a table as shown in FIG. 4 and configured so that a voltage bias based on the depth is superimposed to a small voltage.

With reference to the ultrasound image diagnostic device S configured in the above manner, the principle that a reception signal is amplified by a stochastic resonance phenomenon will be described.

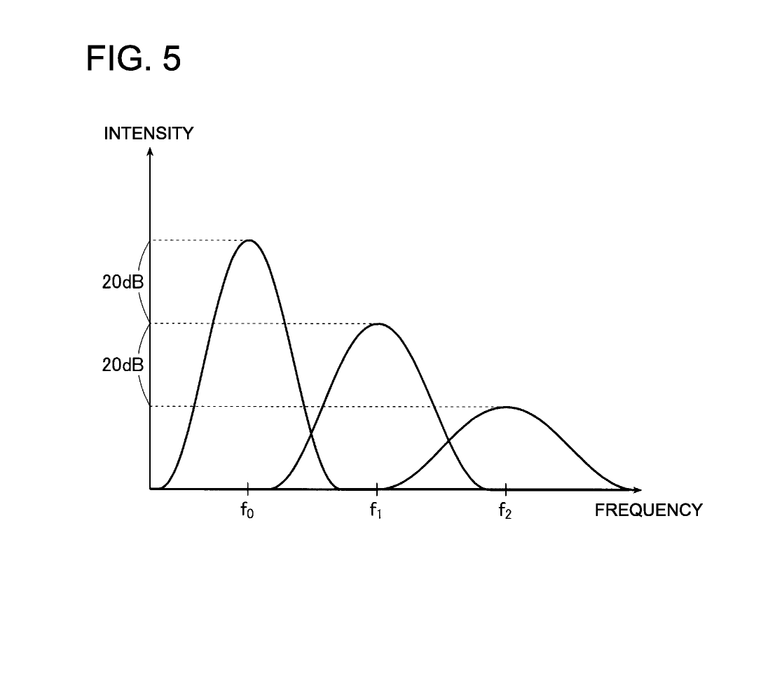

As shown in FIG. 5, reflection ultrasound obtained from transmission ultrasound having been output toward a subject contains a fundamental component whose central frequency is f₀, a second harmonic component whose central frequency is frequency f₁ twice as large as the fundamental component, and a third harmonic component whose central frequency is frequency f₂ three times as high as that of the fundamental component. These harmonic components have wider bandwidth than the fundamental component but the intensity thereof is small. For example, the intensity of the second harmonic is smaller by 20 dB than that of the fundamental component and the intensity of the third harmonic component is smaller by 20 dB than that of the second harmonic component.

When such reflection ultrasound is received by the transducer 2a and converted into a reception signal, the reception signal comes to contain, in addition to a desired signal component obtained from the reflection ultrasound, system noise generated in the ultrasound image diagnostic device S and a speckle generated via interference of an ultrasound scattering wave in the interior of the subject.

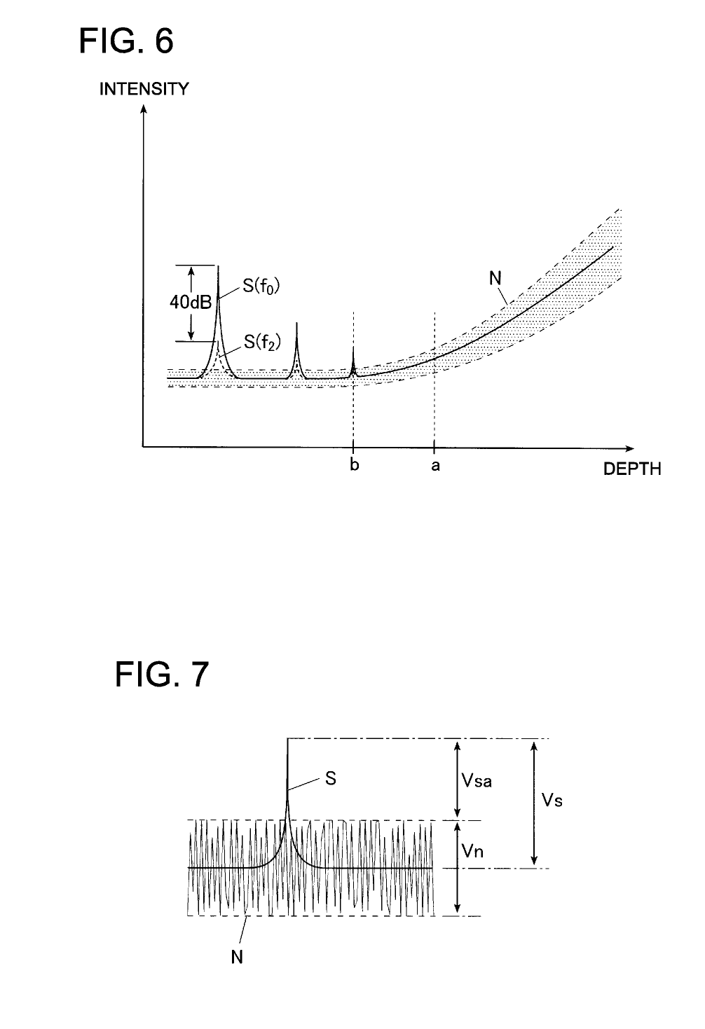

For example, as shown in FIG. 6, a certain reception signal contains, in addition to a desired signal component S, a noise component N such as a system noise component and a speckle component as described above. Herein, in FIG. 6, the range shown by the noise component N represents that in the range, the noise component carries out signal amplification. Further, in the signal component S, S(f₀) represents the fundamental component and S(f₂) represents the third harmonic component.

The signal component S attenuates with an increase in depth. Further, the amplitude of the noise component N (especially, the speckle component) becomes large at a position having a certain level of depth, resulting in an increase in noise influence. Thereby, at a position deeper than depth a, the signal component S is buried in the noise component N and thereby the signal component and the noise component cannot be distinguished. Further, the third harmonic component S(f₂) is smaller by 40 dB than the fundamental component S(f₀) and thereby buried in the noise component N, for example, in a position of depth b smaller than depth a. In this manner, the third harmonic component is characteristic of having excellent spatial resolution and a small number of sidelobes and thereby very useful. However, since the intensity thereof is extremely small, there has conventionally existed the problem of the difficulty of extraction.

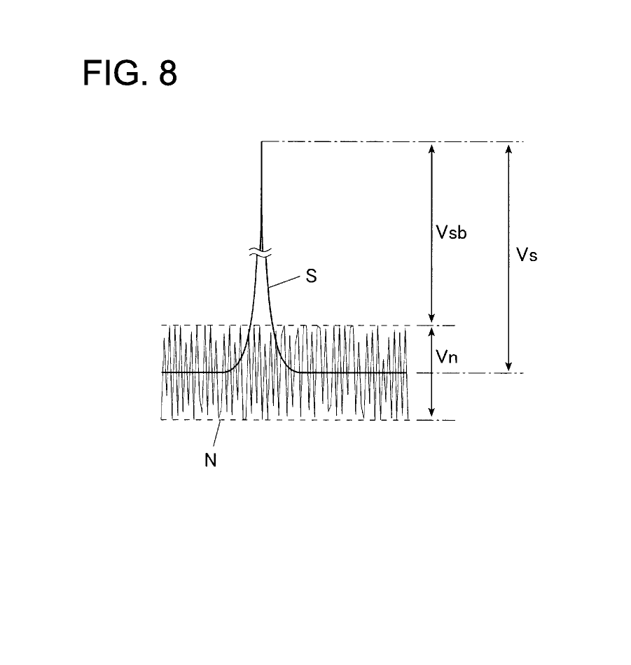

FIG. 7 and FIG. 8 are shown by extracting part of a given reception signal. FIG. 7 represents the relationship between a signal component and a noise component in the case where a reception signal is obtained with no application of a stochastic resonance phenomenon. FIG. 8 represents the relationship between a signal component and a noise component in the case where a reception signal is obtained via the first embodiment.

As shown in FIG. 7, when the intensity of the signal component S at the peak is represented by Vs and the intensity of the noise component N is represented by Vn, S/N can be represented by following Expression (4).

S/N=Vs/Vn (4)

Further, in the signal component S, a portion identifiable in imaging becomes a portion in which the portion buried in the noise component N is eliminated from the intensity Vs of the signal component S. This portion is referred to as a dynamic range in some cases. In FIG. 7, the magnitude thereof is represented by Vsa. Namely as the dynamic range Vsa of the signal component S decreases, the contrast to the noise component N decreases. In an ultrasound image acquired via such a reception signal, the identification of a reflective object in the interior of the subject may become difficult.

In the first embodiment, as described above, since a reception signal is amplified by a stochastic resonance phenomenon, as shown in FIG. 8, the intensity Vs of a signal component S is dramatically increased. On the other hand, the stochastic resonance phenomenon produces just a small influence on the intensity of the noise component N. Therefore, S/N and the dynamic range are dramatically enhanced. Further, as described above, when a noise component superimposed based on the depth is optimized, that is, the pattern of noise superimposed based on the depth is changed, the gain of a harmonic component amplified by the stochastic resonance phenomenon can be allowed to differ with respect to each harmonic order. Accordingly, with regard to the second harmonic component and the third harmonic component whose intensities are extremely small, reception signals having the same intensity as the fundamental component can be obtained.

### Example 1

The present invention will now be detailed by means of examples. However, needless to say, the present invention is not limited to these examples.

### Comparative Example 1

As Comparative Example 1, an ultrasound probe 2 was produced using a transducer 2a, formed of PZT, (the number of elements is 192, in which the dimensions of each element are width 0.2 mm×height 8 mm×depth 0.04 mm, and these elements are arrayed in the azimuth direction). Then, at 4 MHz, a focal point of 95 mm, and a focal sound pressure of 0.2 MPa, transmission ultrasound was transmitted to a given phantom (a model used in the operational test of a device) and then reflection ultrasound from a nylon wire of a diameter of 0.1 mm arranged at the focal position of the transmission ultrasound inside the phantom was received.

In that case, the S/N of the reception signal was as follows: −30 dB at a fundamental central frequency of 4 MHz; −45.6 dB at a second harmonic central frequency of 8 MHz; and −60.8 dB at a third harmonic central frequency of 12 MHz.

### Example 1-1

Next, ultrasound was transmitted and received under the same conditions as in Comparative Example 1 and then during reception of reflection ultrasound, white noise of 0.1 Vrms was applied to each transducer 2a.

In that case, the S/N of the reception signal was as follows: −5 dB at a fundamental central frequency of 4 MHz; −35.6 dB at a second harmonic central frequency of 8 MHz; and −55.8 dB at a third harmonic central frequency of 12 MHz, and gain enhancement was observed in the order of the fundamental wave>the second harmonic>the third harmonic.

### Example 1-2

Further, reflection ultrasound was received under the same noise application conditions except that the white noise was changed to blue noise under the same conditions as in Example 1-1 and the noise intensity was 0.15 V/Hz^{1/2}.

In that case, the S/N of the reception signal was as follows: −20 dB at a fundamental central frequency of 4 MHz; −20.6 dB at a second harmonic central frequency of 8 MHz; and −45.8 dB at a third harmonic central frequency of 12 MHz, and gain enhancement was observed in the order of the second harmonic>the third harmonic>the fundamental wave.

### Example 1-3

Still further, reflection ultrasound was received under the same noise application conditions except that the white noise was changed to violet noise under the same conditions as in Example 1-1 and the noise intensity was 0.3 V/Hz^{1/2}.

In that case, the S/N of the reception signal was as follows: −25 dB at a fundamental central frequency of 4 MHz; −20.6 dB at a second harmonic central frequency of 8 MHz; and −30.8 dB at a third harmonic central frequency of 12 MHz, and gain enhancement was observed in the order of the third harmonic>the second harmonic>the fundamental.

[Results]

When each reception signal obtained by Examples 1-1-1-3 was subjected to imaging via a common signal processing, that is, each processing such as envelope detection, brightness conversion, and DSC, an ultrasound image having excellent spatial resolution and contrast and being useful for diagnosis was able to be acquired, compared to Comparative Example 1 in which no noise was superimposed.

### Comparative Example 2

As Comparative Example 2, an ultrasound probe 2 was produced using a transducer 2a, formed of P(VDF-3FE), (the number of elements is 128, in which the dimensions of each element are width 025 mm×height 10 mm×depth 0.04 mm, and these elements are arrayed in the azimuth direction). Then, at 10 MHz, a focal point of 50 mm, and a focal sound pressure of 0.15 MPa, transmission ultrasound was transmitted to the phantom and then reflection ultrasound from a nylon wire of a diameter of 0.1 mm arranged at the focal position of the transmission ultrasound in the interior of the phantom was received.

In that case, the S/N of the reception signal was as follows: −40 dB at a fundamental central frequency of 10 MHz; −60 dB at a second harmonic central frequency of 20 MHz; and −80 dB at a third harmonic central frequency of 30 MHz.

### Example 2

Next, ultrasound was transmitted and received under the same conditions as in Comparative Example 2 and then during reception of reflection ultrasound, pink noise of 0.06 V/Hz^{1/2} was applied to each transducer 2a.

In that case, the S/N of the reception signal was as follows: −15 dB at a fundamental central frequency of 10 MHz; −35.3 dB at a second harmonic central frequency of 20 MHz; and −55.8 dB at a third harmonic central frequency of 30 MHz, and nearly uniform gain enhancement was observed in every frequency bandwidth.

[Results]

When the reception signal obtained by Example 2 was subjected to imaging via a common signal processing, that is, each processing such as envelope detection, brightness conversion, and DSC, an ultrasound image having excellent spatial resolution and contrast and being useful for diagnosis was able to be acquired, compared to Comparative Example 2 in which no noise was superimposed.

### Comparative Example 3

As Comparative Example 3, an ultrasound probe 2 was produced using a transducer 2a, formed of PZT, (the number of elements is 192, in which the dimensions of each element are width 0.2 mm×height 8 mm×depth 0.04 mm, and these elements are arrayed in the lateral direction). Then, at 4 MHz, as well as multiple focal points (30, 60, and 95 mm) and focal sound pressures of 0.06, 0.14, and 0.2 MPa, respectively, transmission ultrasound was transmitted to the phantom and then reflection ultrasound from a nylon wire of a diameter of 0.1 mm arranged at each focal position of the transmission ultrasound in the interior of the phantom was received.

In that case, the relationship among the S/Ns of the reception signals was as follows: −19.2 dB/30 mm, −38.4 dB/60 mm, and −60.8 dB/95 mm at a third harmonic central frequency of 12 MHz.

### Example 3

Next, ultrasound was transmitted under the same conditions as in Comparative Example 3 and received with applying white noise to each transducer 2a for 150 μsec just after the end of the transmission to acquire the return from the nylon wire target. This white noise is characterized by varying its intensity by time at 1000 Vrms/sec.

In that case, the relationship among the S/Ns of the reception signals was as follows: −5.3 dB/30 mm, −5.4 dB/60 mm, and −5.6 dB/95 mm at a third harmonic central frequency of 12 MHz. Nearly uniform S/Ns were obtained by the increase of the gain based on the depth, resulting in the possibility that amplification of a reception signal per depth by TGC is unnecessary.

[Results]

When the reception signals obtained by Example 3 were subjected to imaging via a common signal processing, that is, each processing such as envelope detection, brightness conversion, and DSC, an ultrasound image having excellent spatial resolution and contrast and being useful for diagnosis was able to acquired, when compared to Comparative Example 3 in which no noise was superimposed.

As described above, according to the first embodiment, the transducer 2a outputs transmission ultrasound toward a subject using a drive signal and also receives reflection ultrasound from the subject in order to output a reception signal. The noise output section 19 applies a voltage containing noise to the transducer 2a to amplify the reception signal by a stochastic resonance phenomenon. The harmonic extraction section 13a extracts a harmonic component from the reception signal. The image generating section 14 generates ultrasound diagnostic image data of the interior of the subject based on the harmonic component extracted from the reception signal. The noise output section 19 applies a voltage containing noise such that a harmonic component is amplified by the stochastic resonance phenomenon to the transducer 2a. As a result, even in large depth, a harmonic component having small intensity can be amplified and thereby a reception signal having excellent spatial resolution and enhanced S/N can be acquired. Further, such realization can be attained employing a conventional piezoelectric material with no influence on frame rate. Still further, the size increase of the device is avoided and also excellent production cost is realized.

Further, according to the first embodiment, the noise output section 19 applies a voltage containing noise such that a third harmonic component is amplified by the stochastic resonance phenomenon to the transducer 2a. As a result, even in large depth, a third harmonic component having high frequency makes it possible to acquire an ultrasound image having further enhanced resolution.

Further, according to the first embodiment, the noise output section 19 applies a voltage containing noise to the transducer 2a so that on the basis of the depth of reflection ultrasound received by the transducer 2a, the gain of the amplification of a harmonic component by the stochastic resonance phenomenon is changed per harmonic order. As a result, an appropriate reception signal can be extracted based on the depth and thereby an ultrasound image having more excellent image quality can be acquired.

Further, according to the first embodiment, the noise output section 19 changes the pattern of noise contained in an applied voltage and thereby the order of a harmonic component amplified by the stochastic resonance phenomenon is changed. As a result, just a simple method makes it possible to extract an appropriate signal component based on the depth.

Further, according to the first embodiment, the noise output section 19 applies a voltage containing noise such that a harmonic component is amplified at the timing when the transducer 2a receives reflection ultrasound from a predetermined depth in the subject to the transducer 2a. As a result, a harmonic component can be efficiently amplified.

Still further, according to the first embodiment, the bias voltage supply section 19a superimposes a bias voltage to a voltage output by the noise output section 19 so that the voltage is matched to the baseline of a reception signal. As a result, a reception signal via the stochastic resonance phenomenon can be efficiently amplified.

Further, according to the first embodiment, the bias voltage supply section 19a changes the magnitude of a superimposed bias voltage based on the depth of reflection ultrasound received by the transducer 2a. As a result, reception signals in various depths can be efficiently amplified.

The description in the embodiment of the present invention is just one example of the ultrasound image diagnostic device according to the present invention with no limitation thereto. The detailed configuration and the detailed operation of each functional section constituting the ultrasound image diagnostic device can be also appropriately modified.

Further, in the first embodiment, noise applied based on the depth was optimized. However, regardless of depth, constant noise is applicable.

Still further, in the first embodiment, to allow a stochastic resonance phenomenon to efficiently act, a small voltage containing a noise component was applied with a bias but a configuration with no bias is employable. Further, a constant bias voltage is applicable.

Furthermore, in the first embodiment, a signal component in a reception signal was amplified by the stochastic resonance phenomenon and a noise component such that the reception signal was entirely amplified was superimposed. However, it is possible to superimpose a noise component such that, for example, only at least part of a frequency component such as a second harmonic component and a third harmonic component is amplified and other frequency components are not amplified. In other words, the following case is employable: a noise component such that only a desired frequency component is amplified is superimposed and thereby, compared to the case of superimposing no noise component, the reception signal is not amplified as a whole.

Next, another ultrasound image diagnostic device according to a second embodiment of the present invention will now be described with reference to the drawings. The ultrasound image diagnostic device according to the second embodiment utilizes an ultrasound image diagnostic device main body substantially same as that of the first embodiment of the present invention. Accordingly, explanation of the ultrasound image diagnostic device main body is omitted. At first, a specific structure of the ultrasound probe according to the second embodiment will be explained.

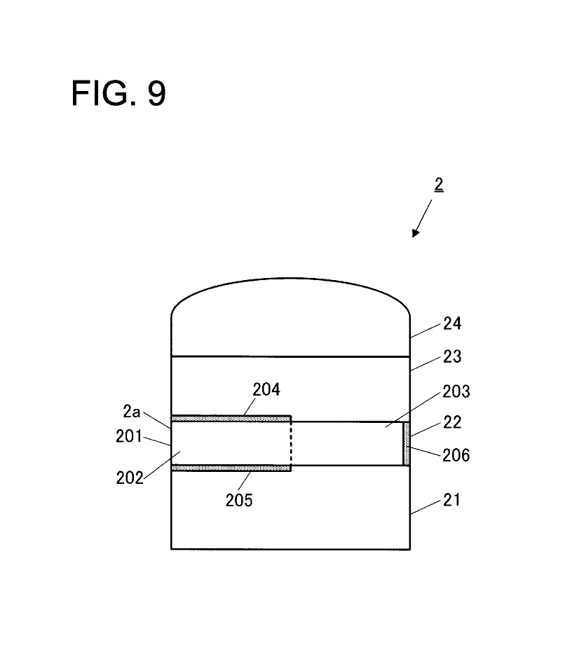

The ultrasound probe 2 according to the second embodiment will now be described with reference to FIG. 9.

The ultrasound probe 2 is configured in such a manner that for example, as shown in FIG. 9, a flat plate-shaped acoustic damping member 21, a piezoelectric section 22 laminated on one main surface of the acoustic damping member 21, an acoustic matching layer 23 laminated on the piezoelectric section 22, and an acoustic lens 24 laminated on the acoustic matching layer 23.

The acoustic damping member 21 is a flat plate-shaped member formed of a material absorbing ultrasound and absorbs ultrasound emitted in the acoustic damping member 21 direction from the piezoelectric section 22.

The material constituting the acoustic damping member 21 includes, for example, a material in which a resin such as an epoxy resin is mixed with acoustic scattering powder. Such a material makes it possible to increase the attenuation rate of ultrasound by an acoustic scattering body.

As the acoustic scattering powder, there can be listed, for example, tungsten (W), molybdenum (Mo), silver (Ag), platinum (Pt), palladium (Pd), indium (In), scandium (Sc), yttrium (Y), and tantalum (Ta). From the viewpoint of cost and availability, in the second embodiment, tungsten is used.

The piezoelectric section 22 is configured in such a manner that a plurality of the above transducers 2a are arrayed each with a predetermined clearance. The piezoelectric section 22 can carry out interconversion between an electrical signal and ultrasound by use of a piezoelectric phenomenon generated by a piezoelectric material possessed by each transducer 2a. The piezoelectric material applied in the second embodiment is, for example, PZT or P(VDF-3FE) with no limitation thereto. Incidentally, the specific configuration of each transducer 2a will be described later.

The acoustic matching layer 23 is a member in which the acoustic impedance of the piezoelectric section 22 and the acoustic impedance of a tested subject are matched. The acoustic matching layer 23 may be configured using a single layer or a plurality of layers. For example, when reception frequency bandwidth is allowed to be larger, the acoustic matching layer 23 is preferably configured using a plurality of layers.

The acoustic lens 24 is a member to converge ultrasound transmitted toward a tested subject and has, for example, convex surface as shown in FIG. 9. Herein, employable are those in which an acoustic matching layer 23 and an acoustic lens 24 are integrally configured.

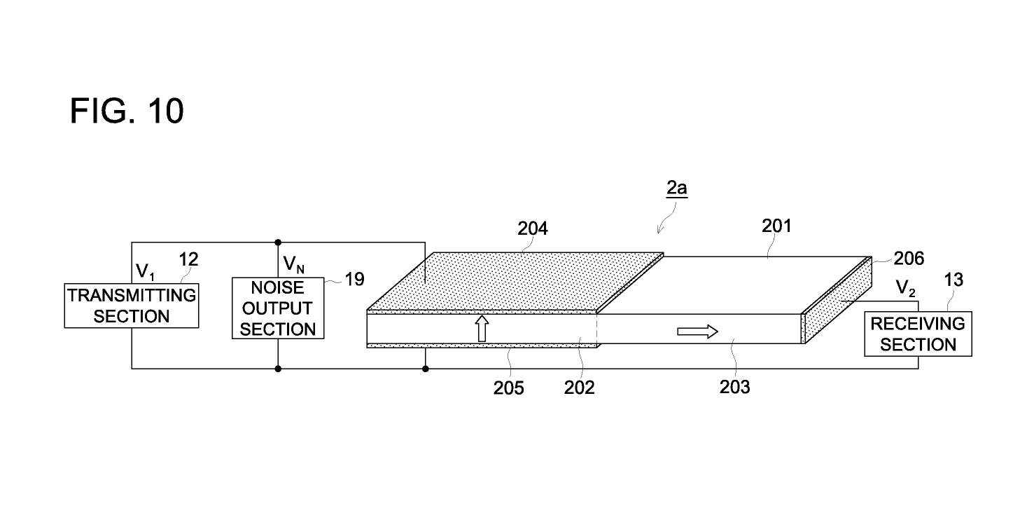

Next, the specific configuration of a transducer 2a constituting the piezoelectric section 22 in the second embodiment will be described with reference to FIG. 10. The transducer 2a according to the second embodiment is configured via application of the configuration of a so-called Rosen-type piezoelectric transformer.

Namely, as shown in FIG. 10, the transducer 2a is provided with a piezoelectric plate 201 as a piezoelectric member formed of a rectangular flat plate-shaped piezoelectric material. In the piezoelectric plate 201, a primary side section 202 and a secondary side section 203 are formed in the longitudinal direction. On the top and bottom faces of the primary side section 202, that is, on the faces intersecting with the thickness direction of the primary side section 202, a pair of primary side electrodes 204 and 205 are provided so as to face each other via the piezoelectric plate 201. Further, on the end face intersecting with the longitudinal direction of the secondary side section 203, a secondary side electrode 206 is provided. The primary side section 202 is polarized in the thickness direction and the secondary side section 203 is polarized in the longitudinal direction. The polarization direction of each of the primary side section 202 and the secondary side section 203 is shown by an arrow in FIG. 10.

The primary side electrodes 204 and 205 are connected to the transmitting section 12 and applied with the transmission voltage V₁ of a drive signal transmitted from the transmitting section 12. In the piezoelectric plate 201, when the transmission voltage V₁ is applied to the primary side electrodes 204 and 205, the primary side section 202 is oscillated in the thickness direction via the inverse piezoelectric effect possessed by the piezoelectric plate 201 to output ultrasound in the thickness direction. Thereby, transmission ultrasound can be transmitted toward the tested subject. Herein, when the transmission voltage V_{I} is applied to the primary side electrodes 204 and 205, the secondary side section 203 is oscillated in the longitudinal direction. The oscillation energy in the secondary side section 203 is converted into an electrical signal via the piezoelectric effect and then an electrical signal of an output voltage V₂ is output from the secondary side electrode 206. The secondary side electrode 206 is connected to the receiving section 13 and then the electrical signal having been output from the secondary side electrode 206 is input into the receiving section 13. Incidentally, in that case, the electrical signal output from the secondary side electrode 206 is not one obtained by reflection ultrasound from the tested subject. Therefore, in the receiving section 13, for example, the input of the electrical signal is not accepted or processing as an invalid signal is carried out.

On the other hand, the primary side section 202 receives reflection ultrasound from the tested subject, and then, when stress in the thickness direction is added by this reflection ultrasound, the stress is converted into an electrical signal by the piezoelectric effect. At this moment, an electrical signal of a conversion voltage V₃ based on the stress having been added to the primary side section 202 is obtained. The secondary side section 203 oscillates in the longitudinal direction based on the electrical signal having been obtained in the primary side section 202. The oscillation energy in the secondary side section 203 is converted into an electrical signal by the piezoelectric effect and then an electrical signal of an output voltage V₂ is output from the secondary side electrode 206 to be input into the receiving section 13. At this moment, the receiving section 13 processes the output voltage V₂ having been input as a reception signal obtained from the reflection ultrasound.

The output voltage V₂ of an electrical signal having been output from the secondary side electrode 206 via reception of reception ultrasound can be represented by following Expression (3). Herein, Qm represents the mechanical Q value of a piezoelectric material applied to the piezoelectric plate 201. Further, k₃₁ represents the electrical-mechanical coupling constant of the piezoelectric plate 201 in the longitudinal direction and k₃₃ represents the electrical-mechanical coupling constant of the piezoelectric plate 201 in the thickness direction. L represents the dimension of the longitudinal direction of the piezoelectric plate 201 and t represents the dimension of the thickness direction of the piezoelectric plate 201. Then, d₃₃ represents the piezoelectric constant of the piezoelectric plate 201 in the thickness direction.

V₂∝(4·Qmk₃₁k₃₃L·V₃·d₃₃)/(π²·t) (3)

In the second embodiment, the above configuration makes it possible to amplify, to some extent, an electrical signal obtained via reception of reflection ultrasound. Thereby, a weak electrical signal obtained from reflection ultrasound of small intensity traveling from a deep portion of the tested subject can be amplified, and compared to an ultrasound image diagnostic device applied with a conventional transducer, S/N can be enhanced.

Incidentally, the dimension L of the longitudinal direction of the piezoelectric plate 201 applied to the second embodiment can be appropriately set, being, however, set to be the same as the wavelength (λ) of oscillation generated in the longitudinal direction of the piezoelectric plate 201 or λ/2 for efficiency, more preferably λ/2.

The dimension of the longitudinal direction of the primary side section 202 affects space resolution and the intensity of an electrical signal obtained by the piezoelectric effect. Namely, with a decrease in the dimension of the longitudinal direction of the primary side section 202, the tomographic section image of an ultrasound image can be allowed to be thin to enhance spatial resolution. However, the intensity of an electrical signal obtained by the piezoelectric effect decreases and then dynamic range decreases. In contrast, with an increase in the dimension of the longitudinal direction of the primary side section 202, the level of an electrical signal obtained by the piezoelectric effect increases and then the dynamic range increases. However, the tomographic section image of an ultrasound image becomes thick and thereby spatial resolution decreases. Therefore, the dimension of the longitudinal direction of the primary side section 202 is preferably set appropriately in consideration of the spatial resolution and the piezoelectric effect.

Further, it is preferable to set the dimension and the opening channel of the azimuth direction of the transducer 2a to realize an opening width so as to obtain adequate azimuth resolution in a deep portion of the tested subject. This makes it possible that the sound pressure per beam unit cross-sectional area at the focal point is enhanced and also a harmonic component is increased, resulting in enhancement of spatial resolution. Still further, the intensity of a signal component obtained from reflection ultrasound comes to increase and thereby the S/N of a reception signal increases. Namely, dynamic range increases and thereby contrast resolution can increase.

In the second embodiment, to further enhance S/N with respect to a weak signal component as buried due to noise even employing the above configuration, the following configuration is applied.

Namely, in the second embodiment, the noise output section 19 is connected to the primary side electrodes 204 and 205 and a small voltage V_{N} transmitted from the noise output section 19 is applied to the primary side electrodes 204 and 205. When the primary side electrodes 204 and 205 are applied with the small voltage V_{N}, the piezoelectric plate 201 responds to noise contained in the small voltage V_{N} and then the primary side section 202 is minutely deformed in the thickness direction by the inverse piezoelectric effect possessed by the piezoelectric plate 201. In this state, the primary side section 202 receives reflection ultrasound and then the stress corresponding to reception of the reflection ultrasound is amplified by the stochastic resonance phenomenon to be converted into an electrical signal of a conversion voltage V₃ in response to the amplified stress. Then, as described above, when the conversion voltage V₃ is amplified, the output voltage V₂ is also increased in proportion thereto and then the intensity of a signal component input into the receiving section 13 is increased. That is, the S/N of a reception signal is enhanced.

In the second embodiment, in some cases, a piezoelectric sensor is configured using a transducer 2a and a noise output section 19.

For example, in the case of application of the stochastic resonance phenomenon, compared to the case of no application of the stochastic resonance phenomenon, the S/N of a reception signal is expected to be enhanced by a factor of 10²-10⁵ by the effective value and the frequency bandwidth of an applied small voltage.

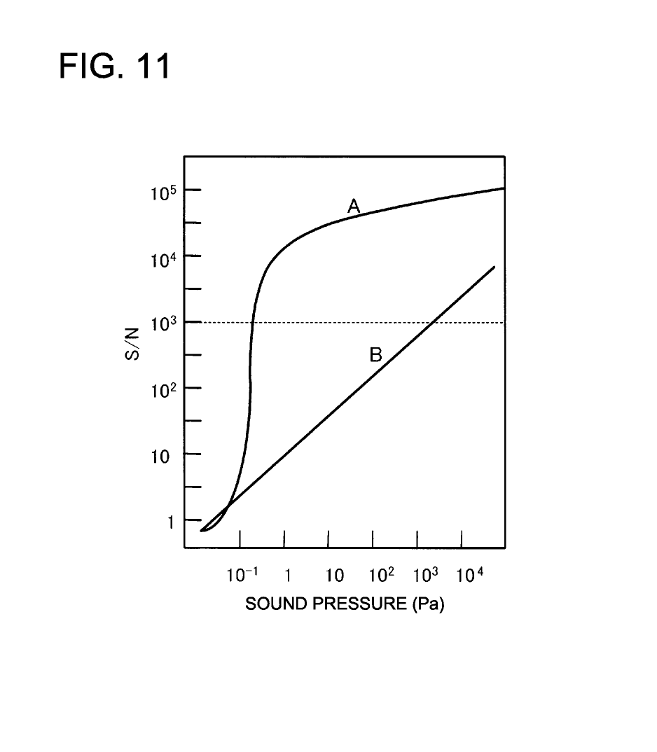

In the case of application of the stochastic resonance phenomenon and in the case of no application of the stochastic resonance phenomenon each in the second embodiment, the detection sensitivity of a reception signal will now be described with reference to FIG. 11. In FIG. 11, the solid line A represents the S/N of a reception signal obtained by the sound pressure of reflection ultrasound against the piezoelectric plate 201 in the case of application of the stochastic resonance phenomenon. The solid line B represents the S/N of a reception signal obtained by the sound pressure of reflection ultrasound against the piezoelectric plate 201 in the case of no application of the stochastic resonance phenomenon.

As shown in FIG. 11, in the case of no application of the stochastic resonance phenomenon, with respect to the sound pressure of reflection ultrasound added to the piezoelectric plate 201, the S/N of a reception signal linearly increases. However, in the case of application of the stochastic resonance phenomenon, the S/N of a reception signal non-linearly increases. Especially in a range where the sound pressure is small, a dramatic increase is noted.

It is obvious that for example, when the dynamic range required for ultrasound image diagnosis using the ultrasound image diagnostic device S according to the second embodiment is allowed to be 60 dB (that is, S/N is 10³), in the case of no application of the stochastic resonance phenomenon, the sound pressure of reflection ultrasound added to the piezoelectric plate 201 needs to be at least about 1000 Pa (10³ Pa), and in contrast, in the case of application of the stochastic resonance phenomenon, the required sound pressure is just about 0.1 Pa (10⁴ Pa). In other words, it is understood that in the case of application of the stochastic resonance phenomenon, the detection sensitivity is enhanced by a factor of about 10⁴, compared to the case of no application thereof.

Further, in the second embodiment, as described above, when a noise component in a small voltage applied to the primary side electrodes 204 and 205 based on the depth is optimized, namely, the pattern of noise superimposed based on the depth is changed, the gain of a harmonic component amplified by the stochastic resonance phenomenon can be allowed to differ per harmonic order. Therefore, also with regard to a second harmonic component and a third harmonic component having small intensity, a signal component having the same intensity as in the fundamental component can be obtained. Further, it is possible that the pattern of noise in a small voltage applied to the primary side electrodes 204 and 205 is appropriately selected to allow a specific frequency bandwidth in a reception signal to be filtered by the stochastic resonance phenomenon.

### Example 4

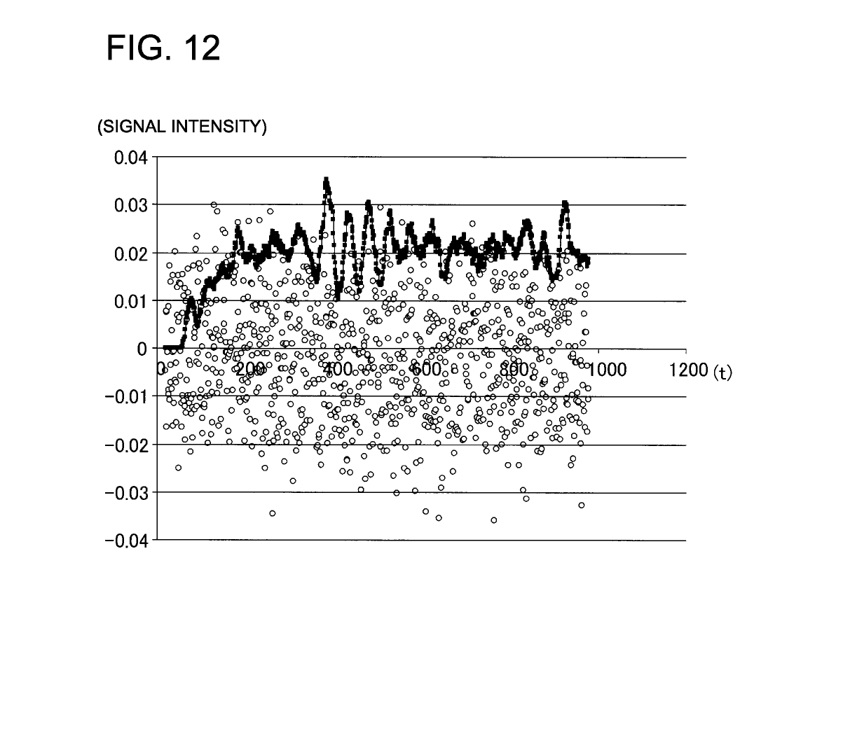

Using the ultrasound image diagnostic device S according to the second embodiment configured in the above manner, ultrasound was transmitted to and received from a tested subject and then the change of the signal intensity of a reception signal was plotted in a time-series manner each in the case of application of the stochastic resonance phenomenon and in the case of no application thereof. The results are shown in FIG. 12. In EXAMPLE 4, as the tested subject, an “Ultrasound 404 GS LE Small Parts Precision Phantom” (produced by Gammex, Inc.) was used. Further, measurement conditions were as follows: the depth of each focal point of transmission and reception was 95 mm and the sampling frequency was 200 MHz; the time when 122 μs had elapsed from the initiation of transmission ultrasound was designated as the sampling initiation point (t=0); and the signal intensity of each sampling point (t=20-979) between 0.1 and 5 μs from the sampling initiation was plotted. In FIG. 12, each plotted point in the case of application of the stochastic resonance phenomenon is represented by a black square and each plotted point in the case of no application of the stochastic resonance phenomenon is represented by a white circle.

As shown in FIG. 12, in the case of no application of the stochastic resonance phenomenon, signal components are buried in noise components and thereby the signal intensities of reception signals are dispersed in a time-series manner. Thereby, it was difficult to identify a reflective body (a pin target) in the interior of the tested subject from the reception signals.

On the other hand, in the case of application of the stochastic resonance phenomenon, the intensities of signal components were amplified and then the reflective body arranged at a depth of 95 mm was confirmed by the reception signals. Namely, S/N enhancement of the reception signals was confirmed.

As described above, according to the embodiment of the present invention, when the transducer 2a is provided with a piezoelectric plate 201 in which a primary side section 202 and a secondary side section 203 are formed in the longitudinal direction, the primary side section 202 is polarized in the thickness direction, and the secondary side section 203 is polarized in the longitudinal direction; primary side electrodes 204 and 205 formed on the face intersecting with the thickness direction of the primary side section 202 of the piezoelectric plate 201; and a secondary side electrode 206 formed on the end face intersecting with the longitudinal direction of the secondary side section 203 of the piezoelectric plate 201, and then stress is added to the piezoelectric plate 201, an electrical signal in response to the added stress is output from the secondary side electrode 206. The noise output section 19 is connected to the primary side electrodes 204 and 205, and then when stress is added to the piezoelectric plate 201, the noise output section 19 applies a small voltage V_{N} containing noise to the primary side electrodes 204 and 205 to amplify the electrical signal by the stochastic resonance phenomenon. Thereby, the S/N of an electrical signal having small intensity generated in response to the stress of a piezoelectric material can be improved.

Further, according to the embodiment of the present invention, when the transducer 2a is provided with a piezoelectric plate 201 in which a primary side section 202 and a secondary side section 203 are formed in the longitudinal direction, the primary side section 202 is polarized in the thickness direction, and the secondary side section 203 is polarized in the longitudinal direction; primary side electrodes 204 and 205 formed on the face intersecting with the thickness direction of the primary side section 202 of the piezoelectric plate 201; and a secondary side electrode 206 formed on the end face intersecting with the longitudinal direction of the secondary side section 203 of the piezoelectric plate 201, and then stress is added to the piezoelectric plate 201 by ultrasound, a reception signal in response to the added stress is output from the secondary side electrode 206. The noise output section 19 is connected to the primary side electrodes 204 and 205, and then when stress is added to the piezoelectric plate 201 by ultrasound, the noise output section 19 applies a small voltage V_{N} containing noise to the primary side electrodes 204 and 205 to amplify the reception signal by the stochastic resonance phenomenon. Thereby, the S/N of a reception signal having small intensity generated in response to the stress of a piezoelectric material can be improved.

Further, according to the embodiment of the present invention, when the transducer 2a is provided with a piezoelectric plate 201 in which a primary side section 202 and a secondary side section 203 are formed in the longitudinal direction, the primary side section 202 is polarized in the thickness direction, and the secondary side section 203 is polarized in the longitudinal direction; primary side electrodes 204 and 205 formed on the face intersecting with the thickness direction of the primary side section 202 of the piezoelectric plate 201; and a secondary side electrode 206 formed on the end face intersecting with the longitudinal direction of the secondary side section 203 of the piezoelectric plate 201, and then a drive signal is provided for the first side electrodes 204 and 205, the piezoelectric plate 201 is oscillated to output transmission ultrasound toward to a tested subject and also when stress is added to the piezoelectric plate 201 by reflection ultrasound from the tested subject, a reception signal in response to the added stress is output from the secondary side electrode 206. The noise output section 19 is connected to the primary side electrodes 204 and 205, and then when stress is added to the piezoelectric plate 201 by reflection ultrasound, the noise output section 19 applies a small voltage V_{N} containing noise to the primary side electrodes 204 and 205 to amplify the reception signal by the stochastic resonance phenomenon. The image generating section 14 generates ultrasound diagnostic image data in the interior of the tested subject based on the reception signal. As a result, the S/N of a reception signal having small intensity generated in response to the stress of a piezoelectric material can be improved. Therefore, an ultrasound image acquired from such a reception signal makes it possible to well carry out ultrasound image diagnosis.

Further, according to the embodiment of the present invention, the harmonic extracting section 13a extracts a harmonic component from a reception signal. The image generation section 14 generates ultrasound diagnostic image data of the interior of a tested subject based on the harmonic component having been extracted by the harmonic extracting section 13a. The noise output section 19 applies a small voltage V_{N} containing noise such that a harmonic component is amplified by the stochastic resonance phenomenon to the primary side electrodes 204 and 205. Thereby, a harmonic component having small intensity can be amplified even in large depth to acquire a reception signal having excellent spatial resolution and enhanced S/N. Further, with no influence on frame rate, the size increase of the device is avoided and excellent production cost is realized.

Further, according to the second embodiment, the noise output section 19 applies a voltage containing noise such that a third harmonic component is amplified by the stochastic resonance phenomenon to the primary side electrodes 204 and 205. As a result, even in a deep portion, a third harmonic component having high frequency makes it possible to acquire an ultrasound image having further enhanced resolution.

Further, according to the second embodiment, the noise output section 19 applies a small voltage V_{N} containing noise to the primary side electrodes 204 and 205 so that the gain of the amplification of a harmonic component by the stochastic resonance phenomenon is changed per harmonic order in response to the depth of reflection ultrasound adding stress to the piezoelectric plate 201. Thereby, an appropriate signal component can be extracted based on the depth and then an ultrasound image of more excellent quality can be acquired.

Further, according to the second embodiment, the noise output section 19 changes the pattern of noise contained in a small voltage V_{N} applied to the primary side electrodes 204 and 205 to change, per harmonic order, the gain of the amplification of a harmonic component by the stochastic resonance phenomenon.

Further, according to the second embodiment, the noise output section 19 applies a small voltage V_{N} containing noise such that a harmonic component is amplified at the timing when stress is added to the piezoelectric plate 201 by reflection ultrasound from a predetermined depth in a tested subject to the primary side electrodes 204 and 205. As a result, a harmonic component can be efficiently amplified.

Further, in the second embodiment, the transducer 2a was formed of a single layer. However, plural layers of transducers 2a may be configured via lamination.

Further, in the second embodiment, the transmitting section 12 and the noise output section 19 were separately configured. However, a configuration in which the transmitting section 12 functions as the noise output section is employable. It is possible that for example, the circuit to transmit a weak signal containing noise and the circuit to transmit a drive signal are the same and a drive signal and a weak signal are output in a switching manner based on the timing.

Further, in the second embodiment, a piezoelectric sensor is configured using the above transducer and noise output section, which is then applied to an ultrasound image diagnostic device. However, this application is employable to other devices with the possible availability of such a piezoelectric sensor.

Furthermore, in the second embodiment, a configuration was employed in which the noise output section 19 was provided for the ultrasound image diagnostic device main body 1. However, a configuration in which the noise output section 19 is provided for the ultrasound probe 2 is employable.

What is claimed is:

1. A method for an ultrasound image diagnostic device comprising a transducer, the method comprising:
outputting, by the transducer, transmission ultrasound toward a subject in accordance with a drive signal;
receiving, by the transducer, reflection ultrasound from the subject, and outputting a reception signal from the transducer;
applying a voltage containing noise to the transducer to amplify the reception signal by a stochastic resonance phenomenon, when the transducer receives the reflection ultrasound;
extracting a harmonic component from the reception signal; and
generating ultrasound diagnostic image data of an interior of the subject based on the extracted harmonic component,
wherein the voltage containing noise is applied to the transducer such that the harmonic component is amplified by the stochastic resonance phenomenon.

2. The method described in claim 1, wherein the voltage containing noise is applied to the transducer such that a third harmonic component is amplified by the stochastic resonance phenomenon.

3. The method described in claim 1, wherein the voltage containing noise is applied to the transducer such that based on a depth of the reflection ultrasound received by the transducer, a gain of an amplification of the harmonic component by the stochastic resonance phenomenon is changed per harmonic order.

4. The method described in claim 3, wherein a pattern of noise contained in the applied voltage is changed, to change, per harmonic order, the gain of the amplification of the harmonic component by the stochastic resonance phenomenon.

5. The method described in claim 1, wherein the voltage containing noise is applied to the transducer such that the harmonic component is amplified at a timing when the transducer receives the reflection ultrasound from a predetermined depth in the subject.

6. The method described in claim 1, further comprising superimposing a bias voltage to the voltage containing noise so that the voltage containing noise matches a baseline of the reception signal.

7. The method described in claim 6, wherein a magnitude of the bias voltage is changed based on a depth of the reflection ultrasound received by the transducer.

8. A non-transitory computer-readable storage medium having a program stored thereon that is executable by a computer of an ultrasound image diagnostic device comprising a transducer, the program being executable to cause the ultrasound image diagnostic device to perform functions comprising:
outputting, by the transducer, transmission ultrasound toward a subject in accordance with a drive signal;
receiving, by the transducer, reflection ultrasound from the subject, and outputting a reception signal from the transducer;
applying a voltage containing noise to the transducer to amplify the reception signal by a stochastic resonance phenomenon, when the transducer receives the reflection ultrasound;
extracting a harmonic component from the reception signal; and
generating ultrasound diagnostic image data of an interior of the subject based on the extracted harmonic component,
wherein the voltage containing noise is applied to the transducer such that the harmonic component is amplified by the stochastic resonance phenomenon.

9. The non-transitory computer-readable storage medium described in claim 8, wherein the voltage containing noise is applied to the transducer such that a third harmonic component is amplified by the stochastic resonance phenomenon.

10. The non-transitory computer-readable storage medium described in claim 8, wherein the voltage containing noise is applied to the transducer such that based on a depth of the reflection ultrasound received by the transducer, a gain of an amplification of the harmonic component by the stochastic resonance phenomenon is changed per harmonic order.

11. The non-transitory computer-readable storage medium described in claim 10, wherein a pattern of noise contained in the applied voltage is changed, to change, per harmonic order, the gain of the amplification of the harmonic component by the stochastic resonance phenomenon.

12. The non-transitory computer-readable storage medium described in claim 8, wherein the voltage containing noise is applied to the transducer such that the harmonic component is amplified at a timing when the transducer receives the reflection ultrasound from a predetermined depth in the subject.

13. The non-transitory computer-readable storage medium described in claim 8, wherein functions further comprise superimposing a bias voltage to the voltage containing noise so that the voltage containing noise matches a baseline of the reception signal.

14. The non-transitory computer-readable storage medium described in claim 13, wherein a magnitude of the bias voltage is changed based on a depth of the reflection ultrasound received by the transducer.
